(19) United States
(12) Patent Application Publication
(10) Pub. No.: US 2022/0411836 A1
(43) Pub. Date: Dec. 29, 2022

(54) METHODS FOR EXTRACTING ANTHOCYANINS TO IMPROVE URINARY HEALTH BY USING CRANBERRIES AND PLANT-BASED LACTOBACILLUS WHICH ENHANCE FEMALE REPRODUCTIVE HEALTH AND EXTRACTS OF THEREBY

(71) Applicant: SAIB & CO Inc., Seoul (KR)

(72) Inventors: JIWON PARK, Seoul (KR); JEONGMIN KIM, Seongnam-si (KR)

(21) Appl. No.: 17/448,751

(22) Filed: Sep. 24, 2021

(30) Foreign Application Priority Data
Jun. 22, 2021 (KR) 10-2021-0080466

(51) Int. Cl. C12P 17/16; A61K 8/49

(52) U.S. Cl. CPC C12P 17/162; A61K 8/498; A61K 2800/85; C12R 2001/25

(57) ABSTRACT

The present invention relates to a method of extracting a low-molecular-weight proanthocyanidin using cranberries and lactic acid bacteria, and more particularly, the method may include (S1) growing and extracting a seed culture from cranberries, (S2) reinforcing the metabolism of a saccharide by adjusting a metabolic process, (S3) extracting the seed culture whose metabolic process is adjusted and culturing the extracted result, (S4) mixing cranberries, distilled water and a saccharide with the grown seed culture and fermenting the mixture, and (S5) extracting an unbound polyphenol from the fermented mixture.

### CROSS-REFERENCE TO RELATED APPLICATION

This application claims priority to and the benefit of Korean Patent Application No. 10-2001-0080466, filed on Jun. 22, 2021, the disclosure of which is incorporated herein by reference in its entirety.

### BACKGROUND

### 1. Field of the Invention

The present invention relates to a method of extracting an anthocyanin component and an extract obtained thereby, and more particularly, to a method of extracting an anthocyanin component for improving urinary health using plant-based lactic acid bacteria enhancing female reproductive health and cranberries and an extract obtained thereby.

### 2. Discussion of Related Art

In the global cosmetic market, the market related to products with an antioxidative function accounted for KRW 130 billion (annual average growth rate of 6.4%) of the total market size in 2018. It is expected that, due to environmental pollution and aging, the antioxidant cosmetic market is expected to continue to expand, and grow to about KRW 220 billion by 2026.

Along with the expansion of the market for cosmetics with an antioxidative function, the demand for antioxidant materials is also increasing. However, particularly, anthocyanin-based materials extracted from mulberries and grape skin are widely used in foods and cosmetics for the purpose of antioxidant functionality, the size of the anthocyanin market used in Korea is close to KRW 32 billion (ire 2018), and as the market grows, the size is continuously expanding.

However, currently, since most of the anthocyanin-based materials used in Korea depend on imports, domestic cultivation of aronia and blueberries and anthocyanin extraction using the same have been tried to lower the dependency on imports.

However, due to high production costs of such a material and the poor quality of the corresponding material, it is still difficult to completely replace an imported material, so there is an urgent need to develop a domestic alternative material with high production efficiency.

In addition, currently, most anthocyanin-based materials are sensitive to light, heat, acidity, etc., and thus have limitations in stability and absorption rate, and. particularly, are sensitive to changes of pH, so a color change easily occurs at a pH 4 or more. In addition, the anthocyanin-based materials have disadvantages of a low absorption rate as a cosmetic material by being bound to sugar, and mostly having a high-molecular-weight structure.

Therefore, the present invention aims to achieve domestic self-sufficiency to lower import dependency of the conventional material, and was invented in the process of overcoming the limitations in stability and absorption rate of an anthocyanin-based material during the self-sufficiency process and developing a material capable of being effectively used as a cosmetic material.

### RELATED ART DOCUMENTS

### Patent Documents

(Patent Document 1) Korean Patent Publication No. 10-1962570 B1, titled “Improved Antioxidant Beverage Composition of Containing Aronia Fermented Product by Lactic Acid Bacteria and Preparation Method of the Same” (registered in Mar. 20, 2019)

(Patent Document 2) Korean Patent Publication No. 10-1045310 B1, titled “Method for Manufacturing Cosmetic Composition Containing Coffee Extract Fermented by Lactobacillus” (registered in Jun. 23, 2011)

(Patent Document 3) Korean Patent Publication No. 10-2055324 B1, titled “Polyphenol-Enriched Plant Extracts and Producing Method Thereof (registered in Dec. 6, 2019)

### SUMMARY OF THE INVENTION

As described above, the present invention is directed to developing a material which can improve limitations in stability and absorption rate of a conventional material, and can be effectively used as a cosmetic material.

The present invention may provide a method of extracting low-molecular-weight proanthocyanidin using cranberries and lactic acid bacteria, which includes: (S1) growing and extracting a seed culture from cranberries; (S2)extracting and culturing the seed culture whose metabolic process is adjusted; (S3) extracting the seed culture whose metabolic process is adjusted and culturing the extracted result; (S4) mixing cranberries, distilled water and a saccharide with the grown seed culture and fermenting the mixture; and (S5) extracting a unbound polyphenol from the fermented mixture.

Preferably, the seed culture in step (S1) is Lactobacillus plantarum.

Preferably, the saccharide in step (S2) is glucose.

Preferably, the fermenting in step (S4) is performed at 38° C. for 38 hours or more.

Preferably, the pH of the final product the fermentation in step (S4) is 3.8, and the number of lactic acid bacteria is 10⁸ CFU or more per 100 g.

Preferably, a content of the non-binding polyphenol in step (S5) is 1.1 g/50 ml.

In addition, the present invention provides a composition including proanthocyanidin extracted according to any of the methods described above.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows a test report for one experimental example of the present invention.

### DETAILED DESCRIPTION OF EXEMPLARY EMBODIMENTS

Hereinafter, exemplary embodiments of the present invention will be described in detail. However, it should be noted in advance that a specific value presented as an example is merely provided to explain the technical idea of the present invention in further detail, in which the technical idea of the present invention is not limited thereto, and various modifications are possible.

In addition, in the specification of the present invention, the same part is denoted by the same reference numeral, and detailed descriptions will be omitted for parts that are known in the art and can be easily created by those of ordinary skill in the art.

1. Research Results

1.1. Proanthocyanidin Extraction Using Lactic Acid Bacteria

The present invention relates to technology for extracting proanthocyanidin using L. Plantarum, and the inventors completed an extraction process and proanthocyanidin extraction.

1.2. Reinforcement of Saccharide Metabolic Function of Lactic Acid Bacteria Through Polymeric Nanobody

L. Plantarum is a species of lactic acid bacteria most commonly found in natural plants. These are beneficial bacteria that can help in maintaining skin and female vaginal health, but is a strain with low viability in competition with other strains and mainly consumes monosaccharides due to its poor metabolic ability.

L. Plantarum has high reactivity and stability with respect to a base material such as cranberries, and there are various bacterial species derived from the base material. However, since most bacteria are killed during washing for a fermentation process, the gist of the present invention is to effectively perform extraction of an effective component by reinforcing the metabolic function of a complex sugar by changing a metabolic process of L. Plantarum using a polymeric nanobody extracted from a plant body, and breaking a bond between glucose and the saccharide in a base material such as cranberries.

1.3. Process of Extracting Active Ingredient (Proanthocyanidin)

An active ingredient (proanthocyanidin) is extracted using a base material such as cranberries, aronia or grape by-products (grape skin and seeds), the base material, L. Plantarum reinforced in sugar metabolic function and purified water are mixed, and fermented at about 32° C. (28 to 38° C.) for about 72 hours (38 to 72 hours). Since a pH value is gradually lowered in the fermentation process, when the pH reaches a predetermined pH range, fermentation may be terminated.

Here, the base material may be any raw material including anthocyanin, in addition to cranberries, aronia or grape by-products (grape skin and seeds), blueberries or acai berries may be included, but the present invention is not limited thereto.

The base materials such as grape by-products and aronia are considerably similar in composition of components of the plants so the above-described anthocyanin extraction method may be applied in the same way, and there may be some differences in the quality or content of the extracted anthocyanin, but the differences can seem to be differences in degree of recognition of identity.

Meanwhile, when the mixed fermented product as described above reaches the pH of about 3.4 (pH 3.4˜3.8), only liquid ingredients were separately extracted from the fermented mixture, and identified by TLC in the extracted liquid, and when cranberries were used, the yield of anthocyanins was confirmed to be about 27.1%.

1.4. Confirmation of Possibility of Proanthocyanidin Depolymerization

The depolymerization of proanthocyanidin extracted in the present invention was confirmed. Specifically, as a result of measurement using H-NMP, UV, HPLC, GPC and TLC, it was confirmed that the proanthocyanidin extracted in the present invention includes a dimer to a tetramer of proanthocyanidin. It can be seen that a high-molecular-weight proanthocyanidin with a polymerization degree of 5 or more, which is difficult to be absorbed in the body, may be processed into a low-molecular-weight proanthocyanidin, and a composite material which has a dimer or tetramer of proanthocyanidin with a molecular weight of about 1,200 or less as a main ingredient can be obtained.

1.5. Confirmation of Material Stability

To use the depolymerized proanthocyanidin according to the present invention in a wider range, it is necessary to confirm its stability. As the corresponding technology relates to a preparation process only using lactic acid bacteria with recognized stability as food and cosmetic raw materials and fruit base materials such as cranberries, it can be safely used since a separate compound or catalyst is not needed like a conventional anthocyanin extraction process.

2. Originality or Point of Difference of the Present Invention

2.1. Domestic Production of Composite Material Using Lactic Acid Bacteria

The present invention relates to technology for extracting proanthocyanidin from cranberries, aronia or grape products using lactic acid bacteria (L. Plantarum), and uses L. Plantarum with reinforced glucose and sugar metabolism in the plant body by modifying a metabolic process using a polymeric nanobody. However, in the case of the present invention, in addition to L. Plantarum, other Lactobacillus species, for example, Lactobacillus lutei, Lactobacillus rhamnosus, and Lactobacillus gassei, can be used.

As a result, the bonds between dietary fiber and glucose in the base material may be broken, and organic acid-binding depolymerized proanthocyanidin may be extracted, and in this process, large amounts of effective ingredients such as L. Plantarum and probiotics and vitamins may be generated, and thus can be used as a composite material with proanthocyanidin.

In this regard, conventionally, there were patents relating to methods of extracting anthocyanin by fermenting blueberries with lactic acid bacteria, but most technologies are for producing simple fermented compounds, and have limitations in that the purity of anthocyanin extracted thereby is too low to have industrial efficiency (within 2%) and the anthocyanin has a high molecular weight, and thus is impossible to be absorbed into the skin or mucous membrane.

In addition, in the patent relating to the “Method of Preparing Proanthocyanidin Oligomer” (inventor: Takashi Tanaka et al.), a method of producing depolymerized proanthocyanidin by a method such as heating with an acidic solution or leaching with ethanol is suggested. However, the method of separately extracting a depolymerized proanthocyanidin-based ingredient was very limited and shows a poor yield, and thus it was difficult to be used the material resulting therefrom.

Therefore, in the present invention, a technology for making proanthocyanidin into a low-molecular-weight material through modification of the metabolism of lactic acid bacteria, and allowing by-products (organic acid, Lactobacillus, etc.) obtained from the production process to have additional functionality as a composite material. More specifically, since the anthocyanidin material according to the present invention becomes a product formed by combination of lactic acid bacteria (Lactobacillus) and an organic acid, in addition to the antioxidative function that the anthocyanidin material normally has, it may also have health functionality (the function of improving intestinal and vaginal health) of Lactobacillus.

2.2. Improvement of Absorption Rate with Depolymerized Proanthocyanidin

As a result of measurement using H-NMP, UV, HPLC, GPC and TLC, it was confirmed that the proanthocyanidin extracted by the technology according to the present invention includes a dimer or a tetramer of proanthocyanidin.

TABLE 3
Proanthocyanidin oligomer

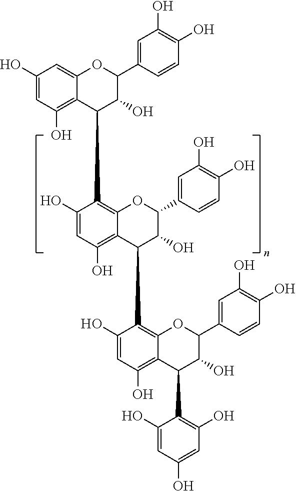

This may allow a high-molecular-weight proanthocyanidin with the degree of polymerization of 5 or more, which is difficult to be absorbed into the body, to be processed into a low-molecular-weight material which is easy to use, and a composite material including a dimer or tetramer of proanthocyanidin as a main ingredient and having a molecule weight of about 1,200 or less may be obtained.

2.3. Economical Production Using Plant By-Products

The technology according to the present invention can produce an economical antioxidant material since it can use food by-products remaining after production of juices or other foods. For example, it is possible to use cranberries, grape, aronia and by-products thereof, which contain a large amount of anthocyanidin, and also possible to use by-products remaining after production of cranberry, grape, and aronia juices and other foods.

Since anthocyanin-based materials are traded at a high price of KWR 3 million per kg (based on a content of 25%), they are difficult to be widely applied to foods and cosmetics.

However, the present invention can use plant by-products, can extract an anthocyanin-based ingredient only by a simple fermentation process, and is expected to enable economical production compared with conventional materials.

3.1. Progression of Experiment According to the Present Invention

3.1.1. Extraction of Seed Culture

A seed culture belonging to Lactobacillus plantarum was cultured and extracted in cranberries.

3.1.2. Adjustment of Metabolic Process of Microorganisms

The metabolism of glucose is reinforced by adjusting the metabolic process of lactic acid bacteria. In general, most lactic acid bacteria can smoothly metabolize monosaccharides or disaccharides, so it is common to add a large amount of saccharides during fermentation or bacterial growth, but in the case of relatively large glucose, metabolic capacity is not high. Therefore, the main object of the present invention is to extract anthocyanin-based polyphenols by degrading a glucose structure in cranberries or grapes with lactic acid bacteria, and thus smoothly extract a target material by reinforcing the glucose metabolic capacity of lactic acid bacteria.

A polymeric nanobody with a size of about 1 to 10 micrometers is generated using a base material with a potent polyphenol production mechanism such as cranberries and young leaf plants by lyophilizing a base material and jet milling the dried result. Here, to maximize the glucose metabolic capacity, the optimal proportion of the base material is calculated.

As described above, since the extraction of anthocyanin-based polyphenols is one of the main objects of the present invention, to this end, the metabolic process of lactic acid bacteria is adjusted, and since the adjustment of the metabolic process of lactic acid bacteria is accomplished according to the principle of exchanging RNA of the plant body with RNA of lactic acid bacteria in the process of introducing a base material powder processed into a polymeric nanobody into lactic acid bacterial cells, it may be difficult for a base material with a weak anthocyanin (or polyphenol) production mechanism to obtain a significant effect in RNA exchange. Therefore, in the present invention, a base material with a strong polyphenol production mechanism is used.

Meanwhile, a seed culture, a polymeric nanobody, purified water, a base material and saccharides were mixed to amplify the cell count of the seed culture. Here, the cell count is preferably 3 billion colony-forming units (CFU) or more per 100 g of the mixture. Although this is an effective number for the fermentation process to be performed sufficiently, a fermentation process is possible with less than 3 billion CFU of the bacteria, but in this case, the process may become too complicated, or the result level of a final product may not be guaranteed.

In the process of metabolizing a polymeric nanobody by a seed culture, the glucose metabolic capacity of the conventional seed culture is reinforced through RNA data exchange.

3.1.3. Seed Culture

A seed culture was extracted and grown for proanthocyanidin extraction, and specifically, the number of lactic acid bacteria may be increased by isolating the liquid part of the mixture containing a large amount of lactic acid bacteria and adding a saccharide thereto.

3.1.4. Proanthocyanidin extraction and Depolymerization

3.1.5. Final Product

Cranberries, the seed culture, distilled water and a saccharide are mixed and fermented. The fermentation temperature is about 38° C. (28 to 38° C.), the fermentation time is 38 hours or more (38 to 72 hours), the pH of the final product is about 3.8 (3.4 to 3.8), and the number of lactic acid bacteria is 10⁸ CFU or more per 100 g.

In the process of proliferating the characterized seed culture in cranberries, glucose degradation and depolymerization of proanthocyanidin were performed.

Generally, since a large amount of vitamins, sugars and glucose are linked to polyphenol produced in plants, which forms a composite, anthocyanin may be generally extracted using ethanol by breaking this bond. However, in this case, a decatalytic process for removing ethanol may be required, and the anthocyanin losing a sugar bond may be easily degraded by light or temperature. However, the present invention uses lactic acid bacteria with reinforced sugar metabolism, and in the process of metabolizing sugars in the base material such as cranberries, the bond between anthocyanin and a sugar may be eliminated, and in this process, depolymerization of the anthocyanin may occur.

4. Test Results According to the Present Invention

4.1. Sample Preparation

The process of preparing a sample in order to conduct an experiment according to the present invention is as follows.

TABLE 4
Sample preparation process

\textcircled{1} Frozen cranberries are prepared.
\textcircled{2} Lactobacillus with a modified metabolic process,
a saccharide and purified water were mixed.
[Mixing ratio of base material]
\textcircled{3} A pH is adjusted to 00 (3.4~3.8) by fermentation
at 00° C. (28 to 38 C) for 00 hour (38 to 72 hours).
Stirring and extraction are conducted.

4.2. Comparative Experiment for Confirming whether Lactic Acid Bacteria are Suitable for Extraction of Low-Molecular-Weight Anthocyanin

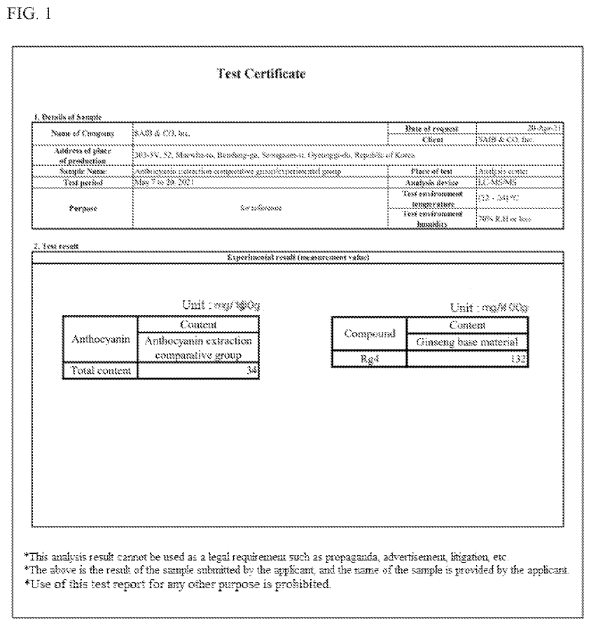

Referring to FIG. 1, in one experimental example according to the present invention, a comparative experiment for the production of a low-molecular-weight anthocyanin of common Lactobacilli and lactic acid bacteria whose metabolic process is adjusted by the related technology was conducted.

TABLE 5
Comparative experiment

| | Comparative experiment | Experiment |
|---|---|---|
| | (sample name: anthocyanin extraction | (sample name: anthocyanin extraction |
| Classification | comparative group) | experimental group) |
| Lactic acid bacteria | Seed culture of common L. plantarum | Seed culture of common L. plantarum |
| used | | whose metabolic process is adjusted |
| | | according to the technology |
| Preparation method | 1) mixing frozen cranberries, saccharide and purified water | |
| | 2) fermenting at 34° C. for 00 hours | |
| | 3) extracting fermented compound | |
| Dominant | Rouxiella sp. Rahnella sp. | Lactobacillus plantarum sp.(dominant) |
| microorganism after | Leuconostoc sp. Pseudomos sp. | Lactobacillus sakei. Enterobacteales |
| fermentation | Enterobacteales etc | etc |
| pH after fermentation | 4.7 | 3.2 |
| Content of anthocyanin | 34 mg/100 g | 132 mg/100 g |

Both experiments were conducted under the same conditions except for the lactic acid bacteria used herein. Through comparison of the corresponding experiment, through this technology, it can be confirmed that the lactic acid bacteria whose metabolic process is adjusted have excellent capability of becoming dominant bacteria in fermentation. In addition, in the case of the seed culture of Lactobacilli whose metabolic process is adjusted, it was confirmed that the seed culture also has more excellent capacity of forming an organic acid and more excellent capacity of extracting anthocyanin.

As described above, the present invention can develop a material which overcomes limitations in stability and absorption rate of a conventional material and is capable of being effectively used as a cosmetic material.

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative. For example, each component described as a single unit may be distributed and implemented, and components described as being distributed may also be implemented in a combined form.

The scope of the present invention is defined by the appended claims and encompasses all modifications and alterations derived from meanings, the scope and equivalents of the appended claims.

What is claimed is:

1. A method of extracting a low-molecular-weight proanthocyanidin using cranberries and lactic acid bacteria, comprising:
(S1) growing and extracting a seed culture from cranberries;
(S2) reinforcing the metabolism of a saccharide by adjusting a metabolic process;
(S3) extracting the seed culture whose metabolic process is adjusted and culturing the extracted result;
(S4) mixing cranberries, distilled water and a saccharide with the grown seed culture and fermenting mixture; and
(S5) extracting an unbound polyphenol from the fermented mixture.

2. The method of claim 1, wherein the seed culture in step (S1) is Lactobacillus plantarum.

3. The method of claim 1, wherein the saccharide in step (S2) is glucose.

4. The method of claim 1, wherein the fermenting in step (S4) is performed at 38° C. for 38 hours or more.

5. The method of claim 1, wherein the pH of the final product of fermentation in step (S4) is 3.8, and the number of the lactic acid bacteria is 10⁸ CFU or more per 100 g.

6. The method of claim 1, wherein a content of the unbound polyphenol in step (S5) is 1.1 g/50 ml.

7. A composition comprising proanthocyanidin extracted according to the method of any one of claims 1 to 6.
